(19) United States
(12) Patent Application Publication
(10) Pub. No.: US 2018/0014991 A1
(43) Pub. Date: Jan. 18, 2018

(54) Bellows Urinal

(71) Applicant: Jessica Caroline Leon, Miami, FL (US)

(72) Inventor: Jessica Caroline Leon, Miami, FL (US)

(21) Appl. No.: 15/212,258

(22) Filed: Jul. 17, 2016

(51) Int. Cl. A61G 9/00; A61F 5/455; A47K 11/12

(52) U.S. Cl. CPC A61G 9/006; A47K 11/12; A61F 5/4556

(57) ABSTRACT

The present urinal's function is to assist a female relieve her bladder independently while in the supine, sitting or standing position by providing an angled opening for urine to collect in the container. The urinal is entirely made of a flexible nonporous waterproof material; allowing for reuse. Its structure is comprised of an expandable and retractable bellows mechanism to facilitate compact storage within a hospital dispensing cabinet or while in the user's possession. The opening is lined with a beveled outer seal. When the urinal's opening is placed over the female perineal area, it extends extra-labially from the anterior labial commissure to the perineal body. By providing an outer seal that is angled horizontally, toward the user, prevention of leakage results in the maintenance of hygiene. The containers shape is rectangular facilitating proper placement by providing a base when used in a horizontal/supine position. The handle is located on the superior portion of the neck, permitting the manipulation of the urinal by either the user or by assistive personnel.

### TECHNICAL FIELD

The current invention relates to a female urinal that is to be operated by the user, independently or with assistance by a second party. It is indicated for an individual in the supine, sitting, standing position. The container consists of an angulated extra-labially opening to conform to the female perineum anatomy while collecting urine into the liquid reservoir. The liquid collecting reservoir has a bellows structure made to expand and retract, facilitating storage.

### DESCRIPTION OF THE PRIOR ART

The female anatomy presents hygiene and subsequent infection complications to women in the supine position. In a home or healthcare setting, a female experiencing mobility difficulties would benefit from a portable urinal in comparison to urinary catheters or bedpans. The female perineum anatomy, as related to the elimination of urine, is comprised of a urethra that is surrounded by the minor and major labia. The minor labia and pelvic floor muscles inhibit the stream of urine to flow in one specific direction. The female urethra is substantially shorter in length, facilitating the entry of bacteria contributing to urinary tract infections.

Medical conditions such as obesity, orthopedic history of the hip and lower extremities and altered mental status render ambulation to the restroom or bedside commode obsolete. Urinary catheter use has been proven to cause hospital acquired infections such as urinary tract infections, posing complications to the patient's health and added cost for treatment. Bedpans are made of plastic, and deflect easily under the patient and allow urine to leak from the woman's perineum area down to her rectum, sacral area and the underlying surface. Urine leakage causes additional bacterial growth if the patient is not cleaned properly. Unsanitary conditions increase the risk of pressure ulcers and or skin breakdown.

Employment or recreational conditions such as military service, trucking or camping may not accommodate a woman's basic needs. It may be difficult to locate a restroom to relieve their bladder, without soiling themselves in the process; therefore a portable urinal is necessary.

Hospital dispensing cabinets hold thousands of supplies needed for patient care leaving limited space. Due to items such as cumbersome urinals and bedpans, dispensing cabinets require frequent restocking to prevent dissipation of other items.

Existing female urinals such as: U.S. Pat. No. 4,769,858 (Gamm, Meunchen, VanSice), U.S. Pat. No. 5,387,205 (Cummins), U.S. Pat. No. 6,021,530 (Davis), U.S. Pat. No. 6,338,166 (Hereford), and U.S. Pat. No. 7,846,143 (Abbato) all display the inability to expand and contract, not allowing for the preservation of storage space for other medical supplies.

U.S. Pat. No. 6,338,166 (Hereford) displays a female urine receptacle that places the bottles opening upward and poorly angled, making it difficult to be used while in a supine position with out experiencing urine leakage toward the user.

U.S. Pat. No. 4,769,858 (Gamm, Meunchen, VanSice) and U.S. Pat. No. 7,846,143 (Abbato) urinals have a circular opening that is not adequately shaped to conform to a woman's perineum anatomy making its use uncomfortable.

U.S. Pat. No. 5,387,205 (Cummins) the urinal's opening is angled in a substantially low position allowing backflow of urine. In addition, its description states that a protuberance molded in the opening is intended to extend into the vaginal canal, making this urinal unnecessarily invasive for the female user.

### BRIEF DESCRIPTION OF THE DRAWINGS

The following invention's structure and function is further outlined in the accompanying detailed drawings:

FIG. 1 is the side view of the female urinal in an expanded position

FIG. 2 is the side view of the female urinal in a contracted position

FIG. 3 is the frontal view of the female showing the container's opening when facing a user

### SUMMARY OF THE INVENTION

The present invention is an improved urinal primarily intended for the female perineal anatomy but may serve both genders. The urinal is to be placed in between the female's legs while the opening is placed over the perineal area, whether the user is in standing, sitting or supine position.

Its ovular funnel like opening with beveled edges is constructed to provide comfort while creating a watertight seal when positioned correctly. The top edge of the opening makes contact with the anterior labial commissure portion while the lower portion rests against the perineal body portion of the female perineal area. The opening's lower edge contains an inverted triangular barrier that assists in the prevention of urine leakage out of the container.

The opening leads to the neck that serves as a spout to transport urine into a rectangular liquid reservoir. Both the opening and the neck are angled toward the user in a linear fashion to ensure adequate positioning is obtained against the perineal area. The neck then angles downward and leads to the deposited urine in to the liquid reservoir. In addition, the handle that allows the user to manipulate positioning of the container stems from the neck portion.

The container's liquid reservoir collects a maximum of 1000 milliliters. It is designed in a rectangular shape with four straight sides, four right angle and adjacent sides unequal in length. The anterior side is used as a base along with the user's underlying surface when the container is in use. The bottom side is used as a base when the container is placed upright. The posterior side permits the handle to hover over it. The liquid reservoir itself is distinct in its design being that it is capable of contracting and expanding due to its bellows mechanism. This mechanism facilitates its storage.

The entire container is created with flexible, nonporous, waterproof material that maintains the container hygienic once washed after use, as well as supporting the user's hygiene and health. The urinal is completely transparent allowing for the visualization, which is substantially important in a healthcare setting. The user or a secondary party such as assistive personnel can operate the urinal.

The Objectives for this Urinal are as Follows:

The urinal is primarily made to cater to the female perineal anatomy while keeping hygiene a priority in a supine, sitting, and standing position.

Although the urinal is mainly considered to be for female use, it could be considered a unisex urinal.

The urinal is shaped to collect urine in a uniform manner preventing the user from soiling herself while voiding.

The compact urinal can be carried on ones person or in a dispensing cabinet while only consuming a small amount space in comparison to existing urinals and bedpans.

This urinal reduces the incidence of urinary tract infections used my invasive procedures such as urinary catheters. It also reduces the incidence of sacral skin breakdown and subsequent pressure ulcers that arise from the leakage of urine when bedpans or adult diapers are used.

### BREAKDOWN

Urinal 10
Bellows 11
Liquid reservoir 12
Right Side 13
Left Side 14
Anterior side 15
Posterior Side 16
Opening 17
Beveled Edge 18
Triangular barrier 19
Neck 20
Snap-on lid 21
Lid connector 22
Handle 23
Top 24
Bottom 25
Four-digit indentation 26
Measuring scale 27

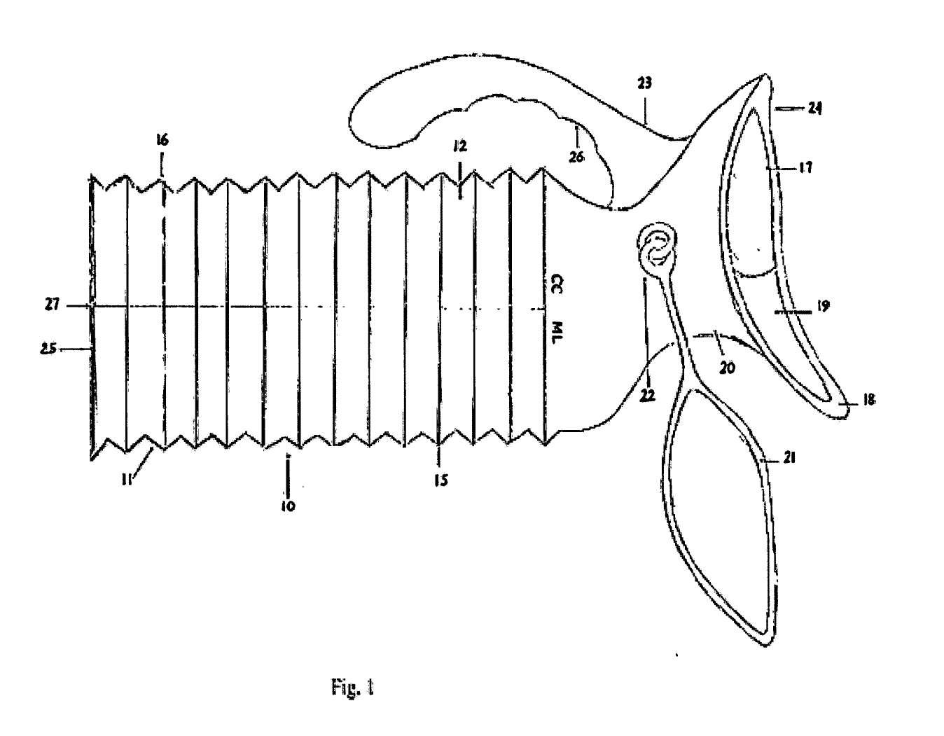

The preferred embodiment discusses in detail the following invention by referencing the drawing FIG. 1-3

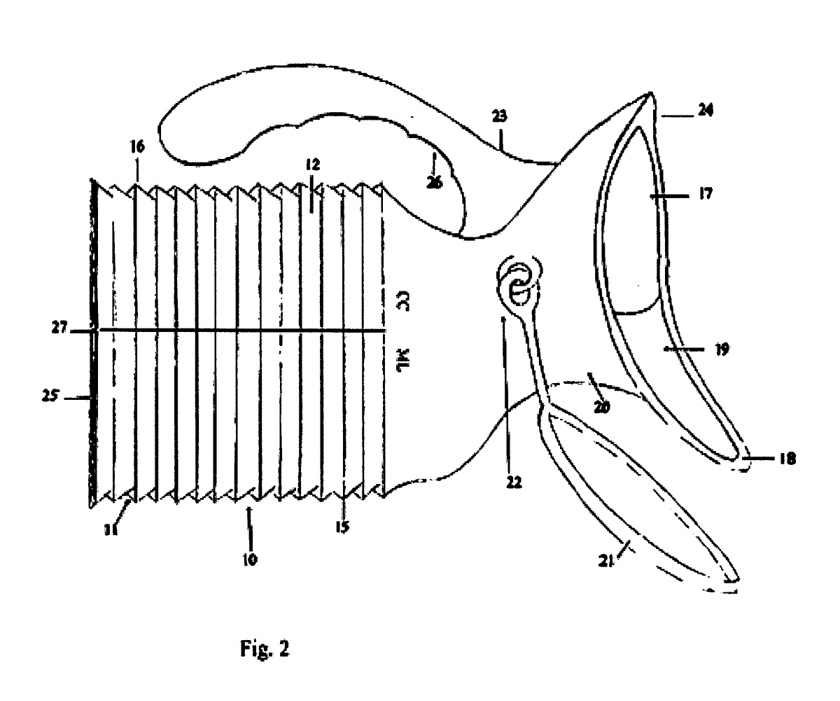

FIG. 1 shows the female urinal 10 in an expanded, hollow state. While the bellows 11 is expanded, it is intended to be used to promote the maximum collection of urine within the container's liquid reservoir 12. FIGS. 1 and 2 show that the urinal consists of an expandable and contractible bellows 11 mechanism that forms a 4-sided rectangular shaped liquid reservoir 12 with four right angles that form rounded corners. The liquid reservoir's sides are as follows: anterior side 15, posterior side 16, right side 13 and the left side 14. In addition to the four sides there are two ends that complete the shape of the urinal 10. The top end begins with the ovular funnel like opening 17 forming into a neck that extends upward and toward the user developing the top 17 end of the urinal. The opposite bottom 25 end is comprised of a thicker material that acts as a base of the urinal 10 when it is placed upright or in a standing position.

The top end of the bottle, as previously stated is comprised of the ovular opening 17 and neck 20 portion. The opening 17 is ovular with beveled edges 18 in order to rest over the perineal area comfortably, between the anterior labial commissure and the perineal body. The lower edge of the opening 17 has an inverted triangular barrier 19 that is intended to act as a precautionary measure, preventing the leakage of deposited urine, outward onto the user and the underlying surface.

The neck 20 is angled upward and laterally, toward the client's perineal area. The neck is angled upward to additionally avoid back flow of urine from the liquid reservoir 12 to the opening. On the left side of the neck 20, the snap-on lid is attached by a connector 22. The snap-on lid 21 is connected to the side of the neck 20 in order to avoid its contact and subsequent interruption of the voiding process when the urinal 10 is in use. Once the user has completed the voiding process but is not yet ready to discard the contents, the lid 21 is simply snapped on to avoid possible odors from escaping the urinal 10.

Posteriorly, the neck 20 is joined with the handle that is designed with a four-digit indentation 26. This aids the user or assistive personnel when manipulating or holding the urinal 10. The handle is located in the central aspect of the neck and extends over the posterior side of the liquid reservoir 12.

FIG. 1 shows that once the urinal 10 is to be used, the anterior side 15 is placed downward against the user's underlying surface if in a supine or sitting position, to gain support. If used while in a standing position, the urinal 10 is simply used vertically permitting the urine stream to follow gravity downward into the liquid reservoir 12.

The entire urinal 10 is constructed of flexible waterproof, nonporous, transparent material allowing for the manipulation and washing of the container as well as visualizing the urine. Visualization and measurement the of the urine proves to be vital in the healthcare setting. The urine may be measured by using the measuring scale 27 located on either side of the liquid reservoir 12.

Because the urinal may be used by both female and male genders, the male is able to use the opening to deposit urine by placing the penis' urinary meatus within it and voiding.

FIG. 2 displays the urinal's 10 bellows 11 mechanism in a contracted state. The bellows 11 mechanism is geared to provide compact storage and is not intended to be used while the urinal 10 is in this state. Compact storage is important when conserving space in many regards. Such examples would be to facilitated storage of other needed healthcare supplies within a dispensing cabinet or the ability to carry the urinal on the user's person for instance in a purse or military transport bag.

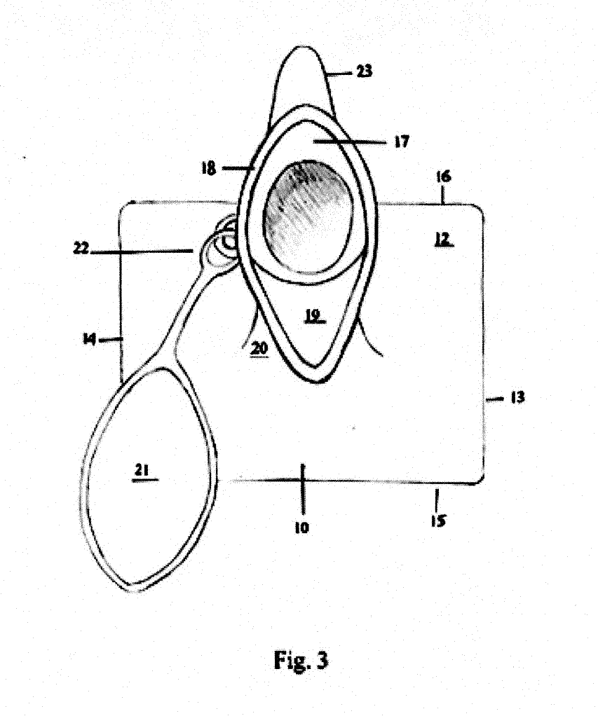

FIG. 3 is the frontal view that the user sees when facing the urinal 10. The ovular shaped opening 17 extends extra-labially over the female perineal area from the anterior labial commissure to the perineal body. It is ideal that the opening 17 is noninvasive as to not introduce additional bacterial to the area or cause physical and/or emotional discomfort. Inaccuracy of the female urine stream is in part due to the anatomical make up of the pelvic muscles and/or labia. For this reason the span in between the top edge and bottom edge of the urinal's 10 opening 17 is meant to cover the previously stated perineal landmarks to accurately capture deposited urine. A beveled edge 18 lining the mouth of the opening ensures comfortable placement. The bottom edge contains an inverted triangular barrier 19 that is placed against the perineal body, located underneath the vaginal orifice. The purpose of this triangular barrier 19 is to impede the leakage urine from the urethra or from deposited urine in the urinal 10.

Although the urinal is primarily invented as a solution to the female voiding process, the present bellows urinal may be used by both male and female genders. It is to be noted that the following disclosed description of each illustration is not limiting to the scope of the invention. The preferred embodiments have been chosen to explain the invention's function when applied and its distinct features that have been referred to in the claims.

1-6. (canceled)

7: A urinal container includes:
A waterproof, transparent, rectangular shaped, reusable reservoir comprised of one solid continuous piece, a superior wall permitting said containers handle to stem from neck portion, an inferior wall that creates a base permitting support when used on a flat surface in a horizontal/supine position while urine is deposited, the milliliter measuring gauge is located on either sidewall of the liquid reservoir and on the left side of the neck the snap-on lid stems, a right side wall, a left sidewall, a thicker bottom or posterior wall that creates a base when said container is placed upright, walls meet creating a 90 degree right angle, a curved ovular opening with an inferiorly positioned triangular barrier impeding urine backflow towards the user, a bellows mechanism comprises the liquid reservoir of said container to expand and contract, said container's structure permits use by both male and female genders.
Four walls; superior, inferior, right and left, comprise the container's expandable and contractible bellows mechanism resembling an extendable straw that in turn creates the liquid reservoir used to collect deposited urine.

8: A container set forth in claim 6 is one solid continuous urinal, comprised of waterproof, nonporous, transparent florescent, flexible material allowing reuse and visualization of the container's location in dark areas.

9: A container set forth in claim 6, includes a handle that hovers over the superior wall, located stemming from the superior portion of the neck and contains a four digit indentation designed for single handed, manual placement and manipulation by either the user or assistive personnel.

10: A container set forth in claim 6, includes a structure comprised of an expandable and retractable mechanism facilitating compact storage when not in use along with a 1000 milliliter measurement gauge located on either side of the liquid reservoir.

11: A container set forth in claim 6, includes a lid that is connected to the left sidewall of the container and is placed over the opening when not in use

12: A urinal container includes:
curved, ovular shaped, funnel, opening angled vertically towards the user's perineum, facilitating the deposit of urine into the urinal container
curved, ovular shaped opening designed for the female anatomy extending extralabially, exteriorly from the anterior labial commissure to the perineal body, a thickened boarder around the said container's opening permits an impermeable seal to be established between the female user and the urinal container, further preventing incidence of urine leakage
a triangular barrier is located in the inferior portion of the curved, ovular opening and is intended to prevent urine backflow towards the user
